(19) United States
(12) Patent Application Publication
(10) Pub. No.: US 2024/0299084 A1
(43) Pub. Date: Sep. 12, 2024

(54) Monitoring Contact Integrity of Reference Electrodes in an RF Neural Ablation System

(71) Applicant: Boston Scientific Neuromodulation Corporation, Valencia, CA (US)

(72) Inventor: Tom Stouffer, Chatsworth, CA (US)

(21) Appl. No.: 18/596,385

(22) Filed: Mar. 5, 2024

Related U.S. Application Data
(60) Provisional application No. 63/488,886, filed on Mar. 7, 2023.

(51) Int. Cl. A61B 18/14; A61B 18/00; A61B 18/16

(52) U.S. Cl. CPC A61B 18/148; A61B 18/16; A61B 2018/00434; A61B 2018/00577; A61B 2018/00875; A61B 2018/00892; A61B 2018/1425; A61B 2018/167

(57) ABSTRACT

A neural ablation system is disclosed, which is coupleable to two reference electrodes affixable to the skin of the patient which act as a return for current provided through a needle electrode that provides the ablation therapy. Because the reference electrodes are included in the ablation current path, they are at risk of heating if they are not well affixed. The system includes resistance monitoring circuitry for monitoring the resistance of the reference electrodes, including a measurement source which operates independently of and is isolated from the ablation source. The measurement source applies a voltage across a series connection of two capacitors. The center node between the capacitors is connected to a common reference of the ablation source. The measurement and ablation sources operate at different frequencies, such that the impedance of the capacitors are different (e.g., open or short circuited) at these frequencies, which provides the isolation.

### CROSS REFERENCE TO RELATED APPLICATIONS

This is a non-provisional application of U.S. Provisional Patent Application Ser. No. 63/488,886, filed Mar. 7, 2023, which is incorporated herein by reference in its entirety, and to which priority is claimed.

### FIELD OF THE INVENTION

This application relates to RF neural ablation systems, and in particular to circuitry and methods for monitoring the contact integrity of reference electrodes used in such systems.

### INTRODUCTION

FIGS. 1A and 1B show a conventional system 10 used to perform Radio Frequency (RF) neural ablation. The ablation system 10 is housed in a chassis which contains system electronics and a user interface 12 to allow a clinician to control the system and to receive system feedback. The chassis includes ports 14 to allow system peripherals to be attached, such one or more RF ablation needles 20. Needles 20 are transcutaneous insertable into the patient proximate to a nerve 24 to be ablated, shown here for example in a patient's forearm 26. RF energy in the form of an AC voltage Vac1 is provided by a high-frequency (HF) RF voltage source 40 within the chassis, which causes an AC ablation current lab to pass to an electrode 22 at the tip of the needle 20. In one example, the frequency of Vac1 can be 480 kHz. Because this needle electrode 22 has a relatively high resistance (R1), it will heat as current passes through it, with the generated heat ablating (essentially destroying) the nerve 24. RF ablation of nerves can be used for a number of different purposes, such as pain management. At lower energies (lower voltages Vac 1), the system 10 can also be used to stimulate nerves, or to temporarily disrupt their operation as is useful in a pain-block scenario. For simplicity, destruction, stimulation, or blocking of nerves are all referred to as “ablation” as used herein. Ablation system 10 can also be used for the ablation of tissue structures other than nervous tissue.

To complete the circuit and allow ablation current lab to flow from the ablation source 40, a reference electrode 30 is also attached to the patient, which is plugged into another of the ports 14 on the chassis. This reference electrode 30 provides a current return through the patient tissue 50. The reference electrode 30 is typically large in area (e.g., several square inches), and may take the form of a patch that is adhered to the patient's skin, and may be pre-formed with a conductive adhesive. The reference electrode 30 is typically adhered to the patient at a distant location from the needle electrode, as in the depicted example is attached to the patient's leg 32. Preferably, the reference electrode 30 has a relatively low resistance (R2). Because the bulk of the patient's tissue 50 between the needle electrode 22 and the reference electrode 30 typically also has a relatively low (R3), most of the resistive heating in the circuit occurs due to the passage of the ablation current lab through the relatively high-resistance needle electrode 22 (R1), as is desired to locally ablate the nerve 24 being treated.

### SUMMARY

An ablation system is disclosed, which may comprise: a first port coupleable to an ablation electrode configured to ablate tissue of a patient; two second ports each coupleable to a different reference electrode affixable to the patient's skin; an ablation source configured to provide an ablation current at a first frequency through the ablation electrode and the reference electrodes; a measurement source configured to provide a measurement current through the reference electrodes; and impedance monitoring circuitry configured to receive a measurement voltage indicative of the measurement current, wherein the impedance monitoring circuitry is configured to determine information indicative of an impedance of the reference electrodes from the measurement voltage.

In one example, the ablation source and the measurement source are configured to be enabled simultaneously to produce the ablation current and the measurement current. In one example, the information indicative of the impedance of the reference electrodes comprises a serial impedance of each of the reference electrodes. In one example, the ablation electrode is configured for transcutaneous insertion in the patient. In one example, the system further comprises a capacitive network provided across the two second ports. In one example, the ablation source is configured to provide the ablation current at a first frequency through the ablation electrode, the reference electrodes, and the capacitive network. In one example, the measurement current does not pass through the capacitive network. In one example, the capacitive network comprises a serial connection of a first and a second capacitor across the two second ports. In one example, the ablation source is coupled to a node between the two serially-connected capacitors. In one example, the measurement source is configured to provide the measurement current at a second frequency through the reference electrodes, wherein the second frequency is different from the first frequency. In one example, the measurement voltage comprises a voltage across the reference electrodes. In one example, the system further comprises at least one resistor in series between the measurement voltage and the measurement source. In one example, the measurement source comprises a current source, and wherein the measurement voltage comprises a voltage across the current source. In one example, the system further comprising a sense resistor in series with the reference electrodes, wherein the measurement voltage is indicative of a voltage across the sense resistor. In one example, the impedance monitoring circuitry comprises control circuitry, wherein the control circuitry is configured to receive information indicative of the measurement voltage. In one example, the information indicative of the measurement voltage comprises digitized versions of the measurement voltage. In one example, the control circuitry is configured with an algorithm to assess the information indicative of the measurement voltage to determine the information indicative of the impedance of the reference electrodes. In one example, the algorithm is configured to compare the information indicative of the impedance of the reference electrodes to a threshold. In one example, the algorithm is configured to disable the ablation source if the information indicative of the impedance of the reference electrodes is greater than the threshold. In one example, the measurement voltage comprises an AC voltage. In one example, the impedance monitoring circuitry comprises a rectifier for producing a DC voltage from the measurement voltage. In one example, the system further comprises a comparator configured to compare the DC voltage to a reference voltage. In one example, the impedance monitoring circuitry is configured to disable the ablation source if the DC voltage is greater than the reference voltage.

A method is disclosed for operating an ablation system, which may comprise: (a) enabling a measurement source of the system to measure a first impedance of two reference electrodes applied to a patient's skin; (b) using the measured first impedance to determine whether the reference electrodes are properly applied; (c) if the reference electrodes are not properly applied, activating a user interface of the system to issue a prompt to a clinician to reapply the reference electrodes, and returning to step (a); (d) if the reference electrodes are properly applied, enabling an ablation source of the system to cause an ablation current to flow through an ablation electrode to ablate tissue of a patient; (e) enabling the measurement source to periodically measure a second impedance of the two reference electrodes while the ablation source is enabled; and (f) when the second impedance is above a first threshold, disabling the ablation source, and returning to step (c).

In one example, in step (b) determining whether the reference electrodes are properly applied comprises determining whether the first impedance is lower than a second threshold, and/or whether the first impedance is higher than a third threshold. In one example, in step (b) determining whether the reference electrodes are properly applied comprises determining whether the first impedance is lower than the second threshold and whether the first impedance is higher than the third threshold. In one example, after step (c), in response to the prompt, receiving an input to the user interface to inform the system that the reference electrodes have been applied. In one example, the method further comprises recording an initial one of the second impedances. In one example, the method further comprises determining the first threshold relative to the initial one of the second impedances. In one example, the first threshold comprises an increase over the initial one of the second impedances. In one example, the increase comprises a percentage of the initial one of the second impedances. In one example, the increase comprises a fixed increase to the initial one of the second impedances. In one example, step (f) further comprises issuing an alarm from the user interface. In one example, the method further comprises disabling the alarm at step (d). In one example, the ablation electrode is transcutaneously inserted in the patient. In one example, the method is performed by an algorithm operating in control circuitry in the system.

A non-transitory computer readable media is disclosed comprising instructions executable in an ablation system comprising two reference electrodes affixable to a patient's skin and an ablation electrode. The instruction when executed may be configured to: (a) enable a measurement source of the system to measure a first impedance of the two reference electrodes; (b) use the measured first impedance to determine whether the reference electrodes are properly applied; (c) if the reference electrodes are not properly applied, activate a user interface of the system to issue a prompt to a clinician to reapply the reference electrodes, and return to step (a); (d) if the reference electrodes are properly applied, enable an ablation source of the system to cause an ablation current to flow through the ablation electrode to ablate tissue of a patient; (c) enable the measurement source to periodically measure a second impedance of the two reference electrodes while the ablation source is enabled; and (f) when the second impedance is above a first threshold, disabling the ablation source, and return to step (c).

### BRIEF DESCRIPTION OF THE DRAWINGS

FIGS. 1A and 1B show a prior art neural ablation system.

FIGS. 2A-2D show a neural ablation system in accordance with an example of the invention. The system includes two reference electrodes, and circuitry and algorithms are provided to monitor the resistance of the reference electrodes, which promotes patient safety during an ablation procedure.

### DETAILED DESCRIPTION

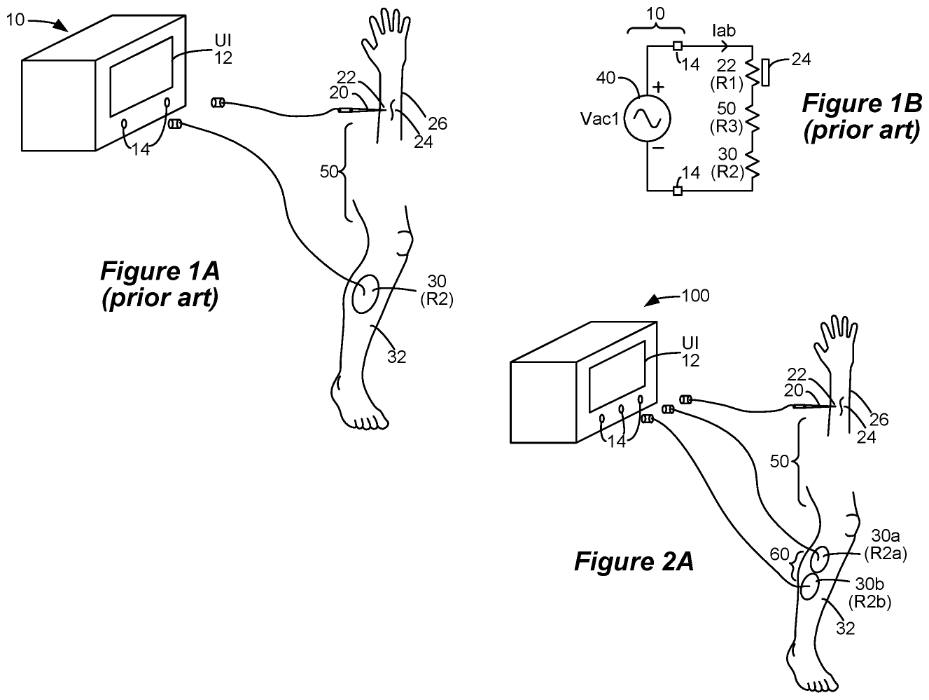

A concern in an RF neural ablation system such as 10 is inadvertent heating at the reference electrode 30. Such inadvertent heating can occur as a result of poor contact of the reference electrode 30 to the patient. This can occur if the reference electrode 30 was not well adhered to the patient initially, or starts to peel away from the patient over time during the ablation procedure. If the reference electrode 30 has poor contact, its resistance (R2, FIG. 1B) risew, and its temperature will increase as the ablation current lab passes through it. This is especially true if the resistance of the reference electrode rises close to the resistance of the needle electrode 22 (R1). The relatively large area of the reference electrode 30 assists in providing a low resistance even if its contact to the patient is or becomes poor, but in cases of extremely poor contact such low resistance cannot be guaranteed. The reference electrode 30 thus runs a risk of burning or discomforting the patient.

Because of such concerns, safety regulations governing RF neural ablation systems require some form of monitoring of the integrity of the contact of the reference electrode 30. In theory, this is possible in the context of a system such as 10. For example, although not shown in FIG. 1B, resistance monitoring circuitry can be used to monitor the resistance of the reference electrode 30 (R2). But measuring resistance in the context of the circuitry of FIG. 1B is not ideal, because the ablation current lab is flowing through the reference electrode 30. This current is variable, and can be changed by the clinician during an ablation procedure (e.g., using the user interface 12), which makes determining the resistance of the reference electrode 30 difficult. Further, the resistance of the reference electrode 30 is in series with other resistances, in particular the bulk resistance of the patient's tissue 50 (R3), which may change over time during the course of an ablation procedure.

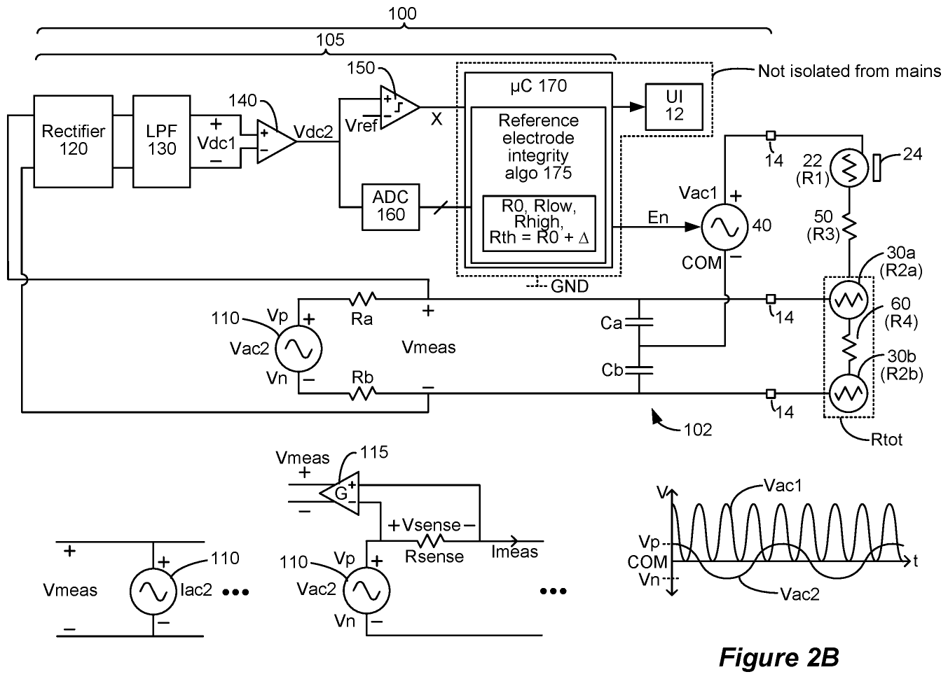

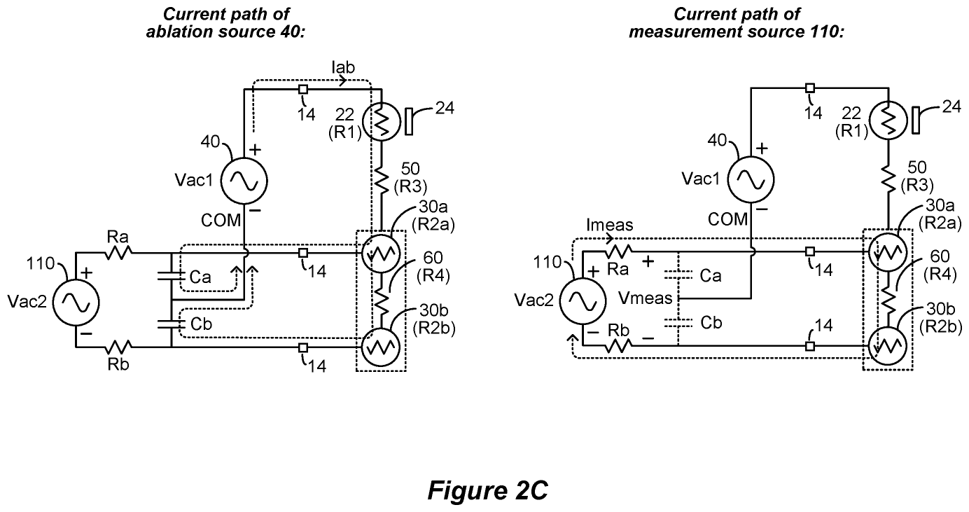

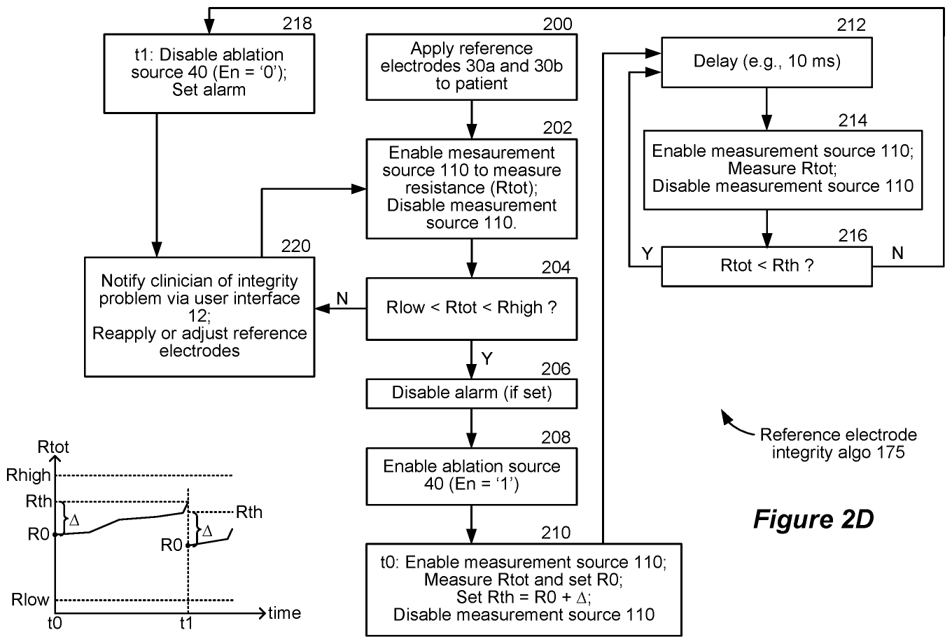

One monitoring scheme which addresses this problem, which is also used in the disclosed examples of the invention, is shown in the system 100 FIG. 2A. In this example, the reference electrode 30 is split into two different reference electrodes 30a and 30b that are adherable to the patient. Reference electrodes 30a and 30b are still relatively large in area and low in resistance when properly adhered (R2a and R2b). Reference electrodes 30a and 30b are preferably adhered to the same general area of the patient, and in some examples can be effectively configured a single reference electrode that has been split (e.g., down the middle) into two halves. As shown, each of the reference electrodes 30a and 30b are connectable to individual ports 14 on the chassis of the system. This isn't however strictly necessary, and instead the reference electrodes 30a and 30b may include wires that meet at a single 2-pin connector couplable to a single 2-pin port 14 on the chassis. While only two reference electrodes 30a and 30b, there may be more than two.

As shown in the circuit diagram of FIG. 2B, the resistance of the electrodes 30a and 30b (R2a and R2b) can be monitored in unison by resistance monitoring circuitry 105, and preferably this occurs independent of the RF ablation therapy that is being provided, i.e., independent of the ablation current lab that passes through the needle electrode 22 as provided by the ablation source 40. In this example, a second RF voltage source 110 is used to provide a bias across the reference electrodes 30a and 30b which allows the resistance of the reference electrodes 30a and 30b to be measured. As will be explained in detail later, the current produced by the measurement source 110, Imeas, is independent of and isolated from the ablation current lab provided by the ablation source 40. Preferably, measurement source 110 provides an AC voltage, Vac2, that is of a different and lower frequency that the ablation source 40 used to provide lab. For example, whereas ablation source 40 provides voltage Vac 1 at a frequency of 480 kHz, measurement source 110 may provide Vac2 at a frequency of 80 KHz.

As shown in FIG. 2B, the system 100 includes a capacitive network 102 comprising capacitors Ca and Cb in series across the ports 14 coupled to the reference electrodes 30a and 30b. The center node between the capacitors Ca and Cb is coupled to the negative input of the ablation source 40, which comprises a common reference, COM. Common reference COM provides a reference for most of the voltages established in the system, and as described later, this common reference COM may be isolated from ground (GND) to provide system isolation. The outer plates of the capacitors Ca and Cb are connected to different ports 14 of the system 100, with each connecting to one of the reference electrodes 30a and 30b. The outer plates of the capacitors are also connected through resistors Ra and Rb to the terminals of the measurement source 110. The capacitive network 102 could in other examples have additional components beyond capacitors Ca and Cb.

The measurement source 110 preferably provides Vac2 centered around the common reference COM and varying between a positive power supply Vp (e.g., +5V to COM) and a negative power supply (e.g., −5V to COM). Vac1 and Vac2 as provided by sources 40 and 110 respectively are shown in a graph provided in FIG. 2B. While these voltages are shown as sinusoidal, one skilled in the art will understand that they may not necessarily be so. In another example, Vac1 and Vac2 can comprise square waves, with the sources 40 and 110 comprising clocked switching circuits.

Isolation between ablation and the reference electrode resistance measurements are provided by the capacitors Ca and Cb, which are set at a value (e.g., 4.7 nF) where their impedance is low at higher frequencies, and higher at low frequencies. Preferably the impedances of capacitors Ca and Cb are significantly different at the two frequencies at which the sources 40 and 110 operate.

FIG. 2C shows the effect of these different impedances. The left figure shows the lab ablation current path established in the system 100 by the higher-frequency ablation source 40. The ablation current Iab proceeds through the needle electrode 22 (R1), the patient's tissue 50 (R3), both reference electrodes 30a and 30b (having resistances R2a and R2b), and to the ports 14 connected to the reference electrodes. Because capacitors Ca and Cb have a low impedance at the higher frequency (e.g., 480 kHz) of the ablation current lab, they act essentially as short circuits to further pass the ablation current lab though both capacitors to the center node between them and to the negative input (COM) of the ablation source 40, which complete the current loop of the ablation current lab. Because the capacitors have low impedance at the frequency of the ablation current lab, notice that the ablation current lab bypasses, and doesn't interfere with, the lower frequency measurement source 110.

The right figure shows the Imeas measurement current path established in the system 100 by the lower-frequency measurement source 110. Because the measurement current Imeas is at a lower frequency, capacitors Ca and Cb act essentially as open circuits. Thus, the measurement current Imeas passes through resistor Ra, to reference electrode 30a through a port 14, through a small portion of the patient tissue 60 between the reference electrodes (R4), to the other reference electrode 30b, to resistor Rb through another port 14, and back to the source 110 to complete the current loop. Significantly, the measurement current in sum passes through the series resistance of the reference electrodes 30a (R2a) and 30b (R2b), and tissue 60 (R4), which in sum comprises a total resistance Rtot that the system monitors (i.e., Rtot=R2a+R2b+R4). Because the reference electrodes 30a and 30b are preferably placed close to one another, the resistance of the intervening tissue 60 (R4) is small and may essentially be negligible, such that Rtot may be approximated as R2a+R2b.

Because capacitors Ca and Cb act essentially as open circuits at the lower frequency of Imeas, this measurement current bypasses, and doesn't interfere with, the ablation current provided by the ablation source 40. In particular, Imeas is isolated from the ablation current lab provided at the needle electrode 22 that ultimately provides the ablation therapy. Imeas does however, like the ablation current lab, pass through the reference electrodes 30a and 30b. However, Imeas is preferably much smaller than lab. As such, Imeas adds only a negligible amount of extra heating (by comparison to lab) at the reference electrodes 30a and 30b, and this allows for monitoring Rtot without adding significant extra risk.

While measurement source 110 is preferably a low-frequency AC source, it could also comprise a DC source (with a frequency of zero).

As noted, the measurement current Imeas produced by the measurement source 110 passes through resistors Ra and Rb. Ra and Rb are preferably equal and may have a resistance of about 500 Ohms in one example. The passage of measurement current Imeas produces an AC voltage Vmeas across the outer plates of the capacitors Ca and Cb, as shown in FIG. 2B. Vmeas is indicative of the resistance (Rtot) of the reference electrodes 30a and 30b, and Rtot can be determined from Vmeas. More specifically, Rtot=Vmeas (Ra+Rb)/(Vac2-Vmeas), where Vac2, Ra and Rb are known. While it is preferred to have resistors Ra and Rb connected to each of the terminals of the measurement source 110 for symmetry, only one of these resistors is required in the depicted example.

Vmeas is provided to the resistance monitoring circuitry 105, and is first provided to a rectifier 120, which like the measurement source 110 can comprise a clocked switched circuit that turns portions of Vmeas positive. The output of the rectifier 120 can be passed to a low pass filter 130, which smooths the output voltage, forming DC voltage Vdc1. As shown, Vdc1 is a differential voltage, which can be presented to a differential amplifier 140 to produce a single-ended voltage Vdc2. The differential amplifier 140 may provide a gain to form Vdc2 as a scaled version of Vmeas. Vdc2, like Vmeas from which it is derived, is indicative of the resistance (Rtot) of the reference electrodes 30a and 30b.

Vdc2 is presented to an analog-to-digital converter (ADC) 160 to digitize Vdc2 as a function of time, with the digitized values of Vdc2 being presented to control circuitry 170 in the system 100. This control circuitry 170 can comprise typical logic circuitry in an electronic apparatus such as a microcontroller, a microprocessor, a programmable logic device (PLD), and combinations of these and similar devices. Control circuitry 170 also includes memory where the digitized Vdc2 values are stored. The control circuitry 170 is also firmware programmed with a reference electrode integrity algorithm 175. As explained in detail below, the algorithm 175 can monitor Rtot during an ablation procedure and disable the ablation source 40 if a high resistance condition indicative of an integrity problem with the reference electrodes 30a or 30b is detected. Control circuitry 170 may comprise part of the impedance monitoring circuitry 105.

Vdc2 is preferably also presented to a comparator 150, where it is compared to a DC reference voltage Vref. Vref is produced by a programmable voltage generator (not shown), and set (by control circuitry 170) to a value corresponding to a high resistance of Rtot clearly indicative of poor contact of one or both of the reference electrodes 30a or 30b. While the system 100 can determine Rtot digitally using reference electrode integrity algorithm 175, comparator 150 is useful as a hardware failsafe to generate a control signal X when a high resistance condition of Rtot is detected, and preferably operates outside of the algorithm 175.

Comparator 150 functions as follows. If either of the reference electrodes 30a or 30b has an abnormally high resistance, Rtot will be high, and Vmeas and Vdc2 will increase, causing X to be set (‘1’) when Vdc2>Vref. Control signal X is sent to the control circuitry 170, which can take immediate action in light of this potentially unsafe condition. (Control signal X may comprise an interrupt). In particular, the control circuitry 170 can disable the ablation source 40 via enable signal En (En=‘0’), which turns off the ablation current lab. This eliminates the risk that lab passing through the high-resistance reference electrodes 30a and 30b will heat and injure the patient. In addition to disabling the ablation source 40, the control circuitry 170 can take other steps. In particular, the control circuitry 170 can use the system 100's user interface 12 to inform the clinician that a high resistance condition has been detected and therefore that the ablation source 40 has been disabled. This can involve the user interface 12 issuing visible notifications, on a screen for example, and/or audible indications.

As noted, the system 100 can also determine the resistance of Rtot of the reference electrodes 30a and 30b using reference electrode integrity algorithm 175. The algorithm 175 continually receives the digital values of Vdc2 as provided by the ADC 160 during the ablation procedure. The algorithm 175 may convert the received Vdc2 samples to resistance values indicative of Rtot. This can involve use of the equation stated earlier, Rtot=Vmeas (Ra+Rb)/(Vac2-Vmeas), where Vac2, Ra and Rb are programmed into the algorithm 175. If necessary, the algorithm 175 can also descale the received Vdc2 values to recover Vmeas, which may be useful if the resistance monitoring circuitry 105 (e.g., the differential amplifier 140) had scaled Vmeas to produce Vdc2 earlier. The graph in FIG. 2D shows the resistance values Rtot determined by the algorithm 175 as a function of time during an ablation procedure.

The algorithm 175 can additionally determine, or be programmed with, a number of values which are shown graphically in FIG. 2D. First, the algorithm 175 can determine R0, the initial resistance of Rtot at the beginning of an ablation procedure (10). As will be seen, the algorithm 175 assumes that the reference electrodes 30a and 30b are well adhered at t0, and hence that Rtot=R0 is suitably low at t0. From R0, the algorithm 175 can determine threshold Rth. Rth is set by the algorithm 175 to be higher than R0, and comprises a threshold wherein the algorithm 175 considers Rtot to be too high and thus indicative of reference electrode integrity problem. Thus, as explained later, if Rtot exceeds Rth, the algorithm 175 will take precautionary actions. Rth can be determined in different manners, but is shown as an increase Δ over R0, i.e., Rth=R0+Δ. This increase Δ can be a percentage of R0 (e.g., 10% of R0) or can comprise a set resistance value increase. Setting Rth relative to R0 is useful because R0 (the initial value Rtot) may vary from patient to patient and depending on their skin properties and the size of the reference electrodes.

The algorithm 175 can additionally determine, or be programmed with, high and low resistance threshold Rlow and Rhigh. The Rlow threshold comprises a value for Rtot that is clearly too low, as may occur if the reference electrodes 30a and 30b are inadvertently touching and are shorted together. The Rhigh threshold comprises a value for Rtot that is clearly too high, as may occur if one or more of the reference electrodes 30a and 30b are completely open circuited. This might occur for example if the leads from the ports 14 to the electrodes 30a or 30b have disconnected. As shown in the graph in FIG. 2D, Rhigh is preferably greater than Rth. The Rlow and Rhigh thresholds can (like Rth) be set relative to R0. However, it is preferred instead that Rlow and Rhigh comprise pre-defined values set by the system manufacturer that are clearly indicative of short or open circuits.

While it can be useful for the algorithm 175 to convert the Vdc2 values to Rtot resistance values, one skilled in the art will understand that this is not strictly necessary, and instead algorithm 175 can operate directly on the unconverted voltages Vdc2 received from the ADC 160. In this regard, the algorithm 175 can be programmed with or determine voltage values or thresholds (e.g., V0, Vth, Vlow, Vhigh) corresponding to the resistance values or thresholds (R0, Rth, Rlow, Rhigh) described earlier.

The graph in FIG. 2D shows an example of Rtot during the course of a particular ablation procedure. In this example, Rtot increases from its initial value R0 (e.g., at time t0), presumably because one or more of the reference electrodes 30a or 30b are peeling away from the patient and are not as well adhered as they were initially. As discussed earlier, this presents a potential safety risk. If Rtot eventually exceeds Rth (e.g., at t1), the algorithm 175 can take certain steps. The actions taken by the algorithm 175 at time t1, as well as other possible actions and steps in the algorithm 175, are discussed next.

FIG. 2D shows details of the reference electrode integrity algorithm 175. This algorithm 175 starts with adhering the reference electrodes 30a and 30b to the patient at a suitable location (200). This step may include the clinician providing input to the user interface 12 to inform the system 100 that the reference electrodes have been applied. The algorithm 175 next checks to see whether the reference electrodes 30a and 30b have suitable integrity, i.e., a suitably low resistance Rtot. The low-frequency measurement source 110 is enabled, and the combined resistance of the reference electrodes (Rtot) is determined, as explained previously (202). After Rtot is determined, the measurement source 110 may be disabled.

The next step inquires whether Rtot is between thresholds Rlow and Rhigh described earlier (204). If not, this suggest that the reference electrodes 30a and/or 30b are not well adhered to the patient-they are for example short circuited or open circuited as explained previously. The algorithm 175 may in this circumstance provide some form of notification to the clinician of the reference electrode integrity problem via the user interface 12 (220). Such notification may comprise visual and/or audible alerts. Visual alerts may include informing the clinician of the detected resistance of Rtot; whether these reference electrodes 30a and 30b appear to be shorted or open circuited; and may include instructions to the clinician that the reference electrodes 30a and 30b should be adjusted or re-applied to the patient. Note that step 204 may alternatively assess only whether Rtot is lower than Rhigh, or whether Rtot is higher than Rlow, and may not necessarily assess Rtot relative to both of these thresholds. Again, this step 220 may include the clinician providing input to the user interface 12 to inform that the reference electrodes have been re-applied. Once re-applied, the algorithm 175 may again enable the low-frequency measurement source 110 to measure Rtot (202).

If and when Rtot of the reference electrodes 30a and 30b is eventually suitable at step 204, the algorithm 175 may disable any reference electrode integrity alarms (206) that had been set earlier in the process (e.g., at step 218, discussed subsequently), and ablation therapy may begin. Thus, the algorithm 175 enable the high-frequency ablation source 40 (En=1′) to allow an ablation current lab to flow though the needle electrode 22 (208). Although not depicted, one skilled will understand that ablation therapy may involve the clinician setting the relevant parameters for therapeutic ablation using the system 100's user interface 12, such as setting the ablation current lab.

The next step (210) enables the measurement source 110 to measure Rtot and to set R0 accordingly, which corresponds to time to on the graph of FIG. 2D. Additionally, the Rth threshold (R0+Δ) can be determined and set at this point, with both of R0 and Rth being stored with the control circuitry 175. After Rtot is measured (and R0 and Rth determined), the measurement source 110 may be disabled. While it is preferable to determine R0 and Rth immediately after ablation therapy has started at step 208 and the ablation current lab is flowing, steps 208 and 210 can also be reversed. That is, R0 and Rth can be determined and stored prior to enabling the ablation source 40.

Thereafter, ablation therapy continues while the resistance of the reference electrodes 30a and 30b are periodically monitored. Preferably, such periodic monitoring occurs frequently, such as every 10 ms, and as a result, step 212 provides a delay of this amount. After this delay, the measurement source 110 is again enabled, Rtot is measured, and source 110 is disabled (214). Thereafter, the current value of Rtot is compared (216) to the Rth threshold determined earlier (210). If Rtot is less than Rth, this indicates that the combined resistance of the reference electrodes 30a and 30b is still adequately low, and therefore that these electrodes still seem have good integrity. Thus, ablation and the monitoring process of Rtot can continue: after a delay (212) Rtot is again measured (214) and compared to Rth (216), and this continues iteratively so long as Rtot is less than Rth. Although not shown in FIG. 2D, each Rtot value as measured can be stored by the control circuitry 170, which allows the system 100 to record Rtot as a function of time.

If Rtot becomes greater than Rth (216), this indicates that the combined resistance of the reference electrodes 30a and 30b is too high, which suggests that at least one of these electrodes may have poor integrity, which risks injuring the patient. This occurrence corresponds to t1 on the graph of FIG. 2D. Because the risk of injury has been detected, the algorithm 175 disables the ablation source 40 (En=‘0’) to stop the flow of the ablation current lab (218). Preferably, this step also includes setting an alarm, which can involve the user interface 12 issuing visible notification on its screen, and/or audible indications.

The algorithm 175 may, in addition to providing the alarm, provide some form of notification to the clinician of the reference electrode integrity problem via the user interface 12 at step 220. Step 220 as described earlier may provide visual and/or audible alerts beyond the alarm condition, such as informing the clinician of the detected resistance of Rtot. Step 220 at this point may also display a graph to the clinician of Rtot as a function of time, similar to the graph shown in FIG. 2D, so that resistance variations of the reference electrodes 30a and 30b can be better understood. As before, step 220 may include instructions to the clinician that the reference electrodes should be adjusted re-applied, with the clinician providing input to the user interface 12 to inform that the reference electrodes have been re-applied.

Once re-applied, the algorithm 175 may again enable the low-frequency measurement source 110 to measure Rtot (202). If and when Rtot of the reference electrodes 30a and 30b is eventually suitable at step 204, the algorithm 175 may disable the alarm (206) set earlier (at step 218), and ablation therapy is reinitiated by once again enabling the ablation source 40 (208). Notice at next step 210 that R0 and Rth are preferably re-determined, and used subsequently to monitor the integrity of the reference electrodes 30a and 30b (steps 212-216). This is preferred because these resistance values may change after the reference electrodes 30a and 30b have been re-applied. The graph in FIG. 2D shows the redetermination of parameters R0 and Rth at time t1.

It should be understood that FIG. 2B shows just one example of resistance monitoring circuitry 105, and other examples of circuitry capable of determining or inferring the resistance of the reference electrodes 30a and 30b are possible, as one skilled in the art understands. The bottom of FIG. 2B shows other alternatives. For example, the measurement source could comprise a current source 110 for providing a known low-frequency AC current lac2 to the reference electrodes. In this example, Vmeas could be measured across the current source 110 (with Ra and Rb removed) and presented to the rectifier 120, which would enable the circuitry to determine the resistance (e.g., Rtot=Vmeas/lac2). In another example, Ra and Rb could be replaced by a low-resistance sense resistor Rsense in the current path Imeas. In this example, a voltage drop Vsense can be measured across the sense resistor which is indicative of current Imeas passing through the reference electrodes (Imeas=Vsense/Rsense). Vsense can then be amplified (by scalar G) if necessary by a differential amplifier 115 to produce Vmeas for presentation to the rectifier 120. Because Vmeas is a function of Vsense, Vsense is a function of Imeas, and Imeas is a function of Rtot, Vmeas is indicative of Rtot, and Rtot can thus be determined from Vmeas as reported to the control circuitry by ADC 160. In all of these cases, the voltage Vmeas received by the resistance monitoring circuitry 105 is ultimately indicative of the measurement current Imeas, which is indicative of the resistance of the reference electrodes 30a and 30b (Rtot) and allows that resistance to be determined.

To this point, it has been assumed that the system 100 as described is used to determine the resistance of the reference electrodes 30a and 30b. However, and especially because AC measurement sources are used, more generally may determining the impedance of the reference electrodes, which may be a complex impedance.

An advantage of the system 100 as disclosed relates to its isolation. While the system 100 is configured to plug into typical power mains (establishing ground, GND), only the control circuitry 170 and user interface 12 elements are referenced to this ground, as shown in the dotted lined box in FIG. 2B. Other aspects of the system are referenced to an isolated common reference, COM. This promotes safety because the patient is also referenced to this common reference. Thus, if the patient were to inadvertently come into contact with power mains (e.g., GND) during his ablation procedure, there would be no inadvertent current paths established in the patient, because the ablation source 40 and the measurement source 110 are referenced differently.

The reference electrode integrity algorithm 175 as described can be provided on a non-transitory computer readable medium, such as within the control circuitry 170 itself, or on an external storage device such as optical, magnetic, or semiconductor disks, which may appears in discrete forms or as incorporated into servers or other similar computer systems. The system 100 may include wireless or wired means for downloading or updating the algorithm 175 into the system 100 from such external storage devices.

Although particular embodiments of the present invention have been shown and described, the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. An ablation system, comprising:
a first port coupleable to an ablation electrode configured to ablate tissue of a patient;
two second ports each coupleable to a different reference electrode affixable to the patient's skin;
an ablation source configured to provide an ablation current at a first frequency through the ablation electrode and the reference electrodes;
a measurement source configured to provide a measurement current through the reference electrodes; and
impedance monitoring circuitry configured to receive a measurement voltage indicative of the measurement current, wherein the impedance monitoring circuitry is configured to determine information indicative of an impedance of the reference electrodes from the measurement voltage.

2. The system of claim 1, wherein the ablation source and the measurement source are configured to be enabled simultaneously to produce the ablation current and the measurement current.

3. The system of claim 1, wherein the information indicative of the impedance of the reference electrodes comprises a serial impedance of each of the reference electrodes.

4. The system of claim 1, wherein the ablation electrode is configured for transcutaneous insertion in the patient.

5. The system of claim 1, further comprising a capacitive network provided across the two second ports.

6. The system of claim 5, wherein the ablation source is configured to provide the ablation current at a first frequency through the ablation electrode, the reference electrodes, and the capacitive network.

7. The system of claim 6, wherein the measurement current does not pass through the capacitive network.

8. The system of claim 5, wherein the capacitive network comprises a serial connection of a first and a second capacitor across the two second ports.

9. The system of claim 8, wherein the ablation source is coupled to a node between the two serially-connected capacitors.

10. The system of claim 1, wherein the measurement source is configured to provide the measurement current at a second frequency through the reference electrodes, wherein the second frequency is different from the first frequency.

11. The system of claim 1, wherein the measurement voltage comprises a voltage across the reference electrodes.

12. The system of claim 11, further comprising at least one resistor in series between the measurement voltage and the measurement source.

13. The system of claim 1, wherein the measurement source comprises a current source, and wherein the measurement voltage comprises a voltage across the current source.

14. The system of claim 1, further comprising a sense resistor in series with the reference electrodes, wherein the measurement voltage is indicative of a voltage across the sense resistor.

15. The system of claim 1, wherein the impedance monitoring circuitry comprises control circuitry, wherein the control circuitry is configured to receive information indicative of the measurement voltage,
wherein the information indicative of the measurement voltage comprises digitized versions of the measurement voltage, or
wherein the control circuitry is configured with an algorithm to assess the information indicative of the measurement voltage to determine the information indicative of the impedance of the reference electrodes.

16. The system of claim 15, wherein the algorithm is configured to compare the information indicative of the impedance of the reference electrodes to a threshold, wherein the algorithm is configured to disable the ablation source if the information indicative of the impedance of the reference electrodes is greater than the threshold.

17. The system of claim 1, wherein the measurement voltage comprises an AC voltage.

18. The system of claim 17, wherein the impedance monitoring circuitry comprises a rectifier for producing a DC voltage from the measurement voltage.

19. The system of claim 18, further comprising a comparator configured to compare the DC voltage to a reference voltage.

20. The system of claim 19, wherein the impedance monitoring circuitry is configured to disable the ablation source if the DC voltage is greater than the reference voltage.
